(19) United States
(12) Patent Application Publication
(10) Pub. No.: US 2009/0203134 A1
(43) Pub. Date: Aug. 13, 2009

(54) REMEDY FOR NERVE CELL REGENERATION

(75) Inventors: Masayo Takahashi, Kyoto-shi (JP); Michiko Mandai, Kyoto-shi (JP); Sotaro Ooto, Kyoto-shi (JP); Fumitaka Osakada, Kyoto-shi (JP)

Correspondence Address: KNOBBE MARTENS OLSON & BEAR LLP, 2040 MAIN STREET, FOURTEENTH FLOOR, IRVINE, CA, 92614, US

(73) Assignee: Kyoto University, Sakyo-ku, Kyoto-shi (JP)

(21) Appl. No.: 11/814,157

(22) PCT Filed: Jan. 17, 2006
(86) PCT No.: PCT/JP2006/300517

§ 371 Date: Jul. 17, 2007

(30) Foreign Application Priority Data
Jan. 18, 2005 (JP) 2005-010189

(51) Int. Cl. C12N 5/06; C07K 14/47

(52) U.S. Cl. 435/375; 530/350

(57) ABSTRACT

Wnt3a proteins represented by SEQ ID NO:2, etc. are used as the active ingredient of remedy for nerve cell regeneration, in particular, remedy for retinal cell regeneration which is useful in treating retinitis pigmentosa, age-related macular degeneration, cone dystrophy and so on.

### TECHNICAL FIELD

The present invention relates to a remedy for nerve cell regeneration.

### BACKGROUND ART

Heretofore, nerve cells such as retinal cells of living mammals have been considered that they cannot regenerate. Recently, it has been reported that Muller cells can acquire properties like those of precursor cells by division in response to an acute disorder, and some of which differentiate into retinal neurons. However, the number of Muller cells to be activated was very small (Non-Patent Document 1).

Wnt is a secretory glycoprotein, or a signaling factor essential to body axis formation and formation of the central nerve system and limbs. In recent years, Wnt3a, one of the Wnt family members, has been reported to be associated with self-duplication of hematopoietic stem cells (Non-Patent Document 2). In addition, Wnt2b has been reported to contribute to proliferation of precursor cells of the ciliary marginal zone and maintenance of undifferentiated status thereof in the chick fetal retina (Non-Patent Document 3).

Further, Wnt-6 has been reported to be therapeutically useful in neurogenic disorders, carcinomas, cardiovascular disorders, strokes, developmental disabilities, and so on (Patent Document 1). Further, Wnt-10a has been reported to be therapeutically useful in neurogenic disorders (Patent Document 2). Besides, a method of enhancing proliferation, differentiation, or maintenance of haematopoietic stem cells/primordial cells with Wnt has also been reported (Patent Document 3). However, involvement of Wnt in neuronal regeneration has not been known in the art.

### [Patent Document 1] JP 2000-060575 A

### [Patent Document 2] WO 99/38966

### [Patent Document 3] WO 98/06747

### [Non Patent Document 1] Proc Natl Acad Sci USA. 2004 Sep. 14; 101(37): 13654-9

### [Non Patent Document 2] Nature 423, 409-414(2003)

### [Non Patent Document 3] Development 130, 587-598(2003)

### DISCLOSURE OF THE INVENTION

An object of the present invention is to provide a remedy to be used for nerve cell regeneration.

The inventors of the present invention have intensively studied for addressing the aforementioned object. As a result, the inventors of the present invention have found that a Wnt3a protein or a Wnt3a signaling activator has an effect of inducing Muller cells to proliferate to and differentiate into retinal cells. Therefore, they have found that neural cells such as retinal cells can regenerate by using the Wnt3a protein, and thus the present invention has been completed.

That is, the present invention provides:

(1) A remedy for nerve cell regeneration, comprising a Wnt3a protein or a Wnt3a signaling activator.

(2) The remedy according to (1), wherein the nerve cell is retinal cell.

(3) The remedy according to (2), wherein the remedy is a therapeutic drug for retinitis pigmentosa, age-related macular degeneration, or cone dystrophy.

(4) The remedy according to any one of (1) to (3), wherein the Wnt3a protein has a sequence of amino acids 25 to 352 in SEQ ID No. 2 or a sequence of amino acids Nos. 25 to 352 in SEQ ID No. 2 with substitution, deletion, insertion, or addition of one or several amino acids and has an ability of regenerating nerve cells.

### BRIEF DESCRIPTION OF DRAWINGS

FIG. 1 is a diagram (photographs) that shows the results of immunostaining with a BrdU antibody which was performed on retinal sections added with control or Wnt3a. INL denotes the inner nuclear layer and ONL denotes the neuroepithelial layer of retina.

FIG. 2 is a graph that shows the percentages of BRdU-positive cells in the retina which was added with control or Wnt3a. GCL (left) denotes the ganglionic cell layer and INL (middle) denotes the inner nuclear layer, and ONL (right) denotes the neuroepithelial layer of retina.

FIG. 3 is a diagram (photograph) that represents the results of immunostaining with a glutamine synthetase (GS) antibody and a BrdU antibody. The arrows indicate portions where both GS and BrdU are positive. GCL denotes the ganglionic cell layer. INL denotes the inner nuclear layer and ONL denotes the neuroepithelial layer of retina.

FIG. 4 is a diagram (photograph) that represents a merged image of the results of immunostaining with glutamine synthetase antibody and BrdU antibody which was performed on a retinal section added with an inhibitor of glycogen synthase kinase 3β. Cells brightly-stained in the middle of the section are BrdU-positive cells.

### DESCRIPTION OF THE PREFERRED EMBODIMENTS

The remedy of the present invention comprises a Wnt3a protein as an active ingredient. The Wnt3a protein is preferably a human or mouse Wnt3a protein, and particularly preferably a human Wnt3a protein. An example of a human Wnt3a protein includes a protein having an amino acid sequence of SEQ ID No. 2. An example of a mouse Wnt3a protein includes a protein having an amino acid sequence of SEQ ID No. 4. The Wnt3a protein may be contained in the remedy of the present invention as a full-length protein (precursor protein) or may be preferably contained in the remedy of the present invention as a mature protein which is generated by cleavage of a signal sequence. The human mature Wnt3a protein may be a protein having an amino acid sequence of amino acids 25 to 352 in SEQ ID No. 2. The mouse mature Wnt3a protein may be a protein having an amino acid sequence of amino acids 25 to 352 in SEQ ID No. 4.

Further, it is easily expected that Wnt3a proteins have sequence variations due to amino acid substitutions or the like depending on their respective biological species in origin. In addition, the ability of the Wnt3a protein to regenerate nerve cell is considered to be not affected even by replacement of an amino acid with another amino acid having analogous properties (i.e., conservative substitution) or deletion of an amino acid at a portion not involved in the activity. As long as the nerve cell regeneration ability is retained, the Wnt3a protein may have an amino acid sequence in which the substitution, deletion, addition, or insertion of one or several amino acids has occurred in the amino acid sequence of SEQ ID No. 2 or 4 or in the amino acid sequence of amino acids 25 to 352 in SEQ ID No. 2 or in the amino acid sequence of amino acids 25 to 352 in SEQ ID No. 4. In this case, the term “several” means preferably 2 to 20, more preferably 2 to 10, and particularly preferably 2 to 5. Also, a protein having high homology, such as not less than 90%, and more preferably not less than 95% homology to the amino acid sequences as described above may be used as long as the protein retains the nerve cell regeneration ability.

The Wnt3a protein may be one purified from a biological source or one chemically synthesized, and preferably one obtained by gene recombination. Further, any Wnt3a protein commercially available may be used.

For obtaining the Wnt3a protein by gene recombination, for example, a DNA having a nucleotide sequence of SEQ ID No. 1 (human wnt3a gene) or SEQ ID No. 3 (mouse wnt3a gene) is introduced into E. coli cells, animal cells, a non-human transgenic animal or the like to express a recombinant protein, followed by purification of the protein. In addition, the protein may not always be a purified one. A partially purified product or cell extract may be used for the detection of interaction. Examples of vectors for introducing the above-mentioned DNA into E. coli include pET vector (Novagen Co., Ltd.) and pGEX vector (Amersham Pharmacia Co., Ltd.), while an example of a vector for introducing the DNA into animal cells includes pcDNA vector (Invitrogen Corporation).

The Wnt3a protein can be used for manufacturing a remedy for nerve cell regeneration. The Wnt3a proteins obtained as described above may be directly used as the remedy of the present invention. Alternatively, a remedy may be obtained by combining a pharmaceutically acceptable carrier with the Wnt3a protein. Examples of the carriers include vehicles, stabilizers, tonicity adjusting agents, and buffers.

The formulations of the remedy of the present invention are not specifically limited. For example, formulations for oral administration specifically include tablets, pills, encapsulated agents, granular agents, syrups, emulsions, and suspension agents.

Formulations for parenteral administrations include ointments, creams, injections, fomentations, liniments, suppositories, eye-dropper, nasal absorbents, trans-pulmonary absorbents, and transdermal absorbents.

In particular, ophthalmological local applications preferably include injections (such as systematic application, intravitreal administration, subretinal application, Tenon's capsule application, and subconjunctival administration), trans keratoconjunctival agents, eye-droppers, and ophthalmic ointments.

Any formulation in solution can be prepared by any conventional method, for example, the Wnt3a protein is usually dissolved in an axenic aqueous solution to be used for injection, suspended in an extract, or emulsified and embed in riposome. The formulation may be prepared by combining any of tonicity adjusting agents (such as sodium chloride, potassium chloride, boric acid, or glycerin), buffers (such as a boric acid buffer solution, a phosphate buffer, or an acetate buffer), solubilizing agents (such as surfactants or cyclodextrin), stabilizers (such as citric acid, ethylene diamine tetraacetic acid, sodium sulfite, or sodium bisulfite), and viscosity-imparting agents (such as synthetic polymers, celluloses, or polyalcohols). In addition, for stabilizing the Wnt3a protein, any additional protein such as human serum albumin may be combined.

When the remedy of the present invention is formulated as eye droppers, any of tonicity adjusting agents such as sodium chloride and concentrated glycerin, buffers such as sodium phosphate and sodium acetate, surfactants such as polyoxyethylene sorbitan monoolate, polyoxyl 40 stearate, and polyoxyethylene hydrogenated castor oil, stabilizers such as sodium citrate and sodium edetate, and preservatives such as benzalkonium chloride and paraben may be used if desired. The remedy may be of any pH as long as it is within a range permitted by ophthalmological formulation, and preferably pH of 4 to 8.

The Wnt3a protein has an effect of regenerating nerve cells such as retinal cells. Thus, it can be widely applied as therapeutic agents for diseases that causes neurodegeneration and neural function disorders. The regeneration of nerve cells can be confirmed using as an index the ability to accelerate the differentiation from precursor cells to nerve cells, for example, based on the expression amount of a marker gene specific to nerve cells at the time of addition of Wnt3a to the precursor cells. For example, the retinal cell regeneration effect (differentiation-accelerating effect) can be confirmed by investigating an increase in cell division of Muller cells or an increase in retinal cells using staining with a Rhodopsin antibody or the like. The Wnt3a protein contained in the remedy of the present invention preferably increases the division of Muller cells not less than 10%, and more preferably not less than 50% as compared to the untreated state.

When the remedy of the present invention is applied for the regeneration of retinal cells, the remedy can be applied as a therapeutic remedy for visual function improvement to the ophthalmology disorder that causes retinal degeneration and visual function disorder. Example of the ophthalmology disorder that causes retinal degeneration and visual function disorder include, in addition to those caused by inflammation or trauma, glaucoma (glaucoma primarium, secondary glaucoma), retinal vascular occlusion, diabetic retinopathy, ischemic optic neuropathy, macular degeneration, retinitis pigmentosa, Leber's hereditary optic neuropathy, Oguchi's disease, angioid streaks, retinal periphlebitis, Eales disease, ischemic eye syndrome, retinal arteriolar macroaneurysm, retinopathy by hypertension, renal disease, or the hemopathy, retinal dystrophy, macular dystrophy, macular oedema, denatured retinoschisis, and cone dystrophy.

The remedy of the present invention is systemically or locally administered. The effective dosage and the number of doses vary depending on dosage formulation, administration route, or the age, weight, disorder to be treated, symptom, or severity of the patient. Generally, 0.001 to 100 mg, and preferably 0.01 to 10 mg per an adult can be administered in a single dose or several divided doses.

On the other hand, a compound that enhances the activity of Wnt3a signaling or a compound that activates the transcription of Wnt3a (hereinafter, these compounds will be referred to as Wnt3a signaling activator) can be used as effective ingredient in the remedy for nerve cell regeneration. The Wnt3a signaling activator may be an inhibitor of glycogen synthase kinase 3β (GSK-3β), which has been known to be associated with the signal pathway of Wnt3a (EMBO J. 2005 Apr 20; 24(8): 1571-83.). Examples of a GSK-3β inhibitor include any of known GSK-3β inhibitors or a compound obtained by screening using GSK-3β activity as an index. The GSK-3β inhibitor may be, for example, AR-A014418 (J. Biol., Chem., Vol.278, No. 46, p45937-45945, 2003). Besides, a physiologically acceptable salt, hydrate, or solvate of such a compound or an analog thereof may be also used. The remedy comprising the GSK-3β inhibitor as an effective ingredient can be formulated by an ordinary method. The applicable disorders include those described above.

### Examples

Hereinafter, the present invention will be described in more detail by way of examples. However, the invention will not be restricted by these examples.

The retina excluding the pigment epithelium was obtained from a 6-week-old adult DA rat (obtained from Shimizu Laboratory Supplies Co., Ltd. (Kyoto)) and a retinal organ culture was then carried out on a Millicell chamber filter. The retinal organ culture was conducted according to a method described in Methods 2002; 28: 387-395 and Brain Res 2002; 954: 286-293. The culture medium used was 50% Minimum Essential Medium +HEPES (Invitrogen Corporation)/25% Hank's solution (Invitrogen Corporation)/25% inactivated horse serum/200 μM L-glutamine/5.75 mg/ml glucose.

Wnt3a (100 ng/ml; R&D Systems) and BrdU (5ng/ml; SIGMA) were added to the culture medium everyday and a frozen specimen was then prepared after 4 days, followed by investigating the presence or absence of BrdU-positive divided cells by an immunological staining with a BrdU antibody.

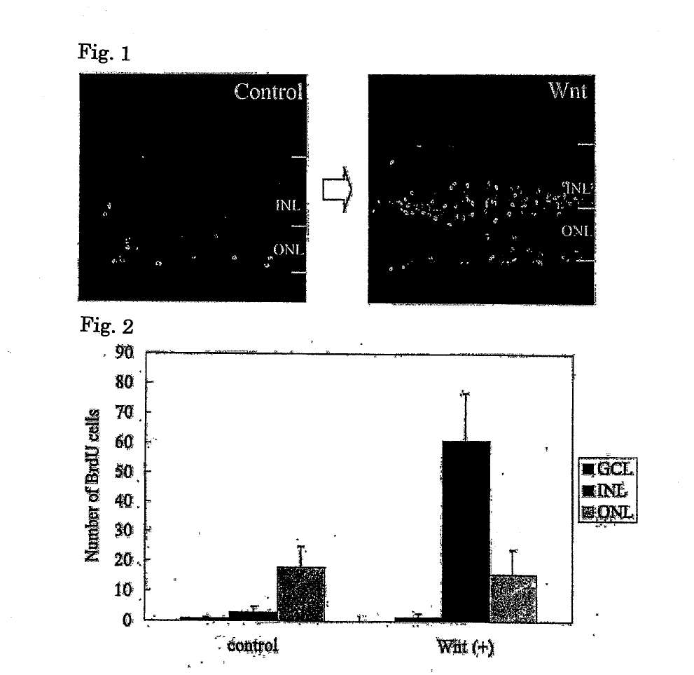

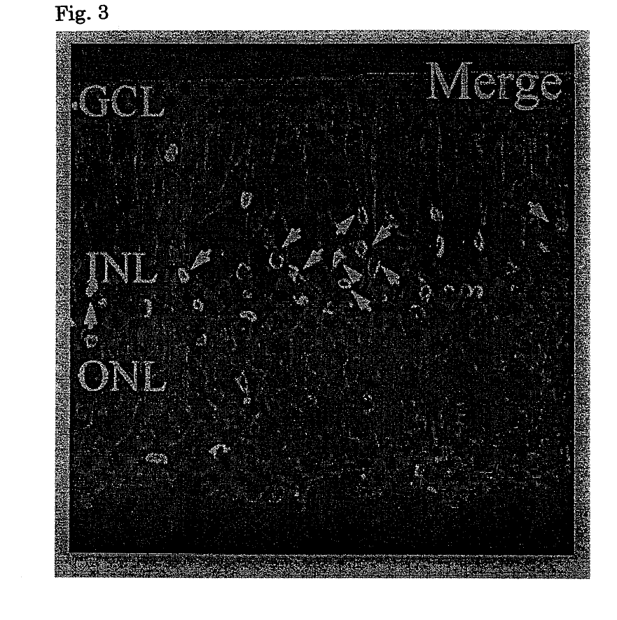

The results are shown in FIG. 1. Compared with the control which did not contain Wnt3a, organ culture added with Wnt3a showed many BrdU-positive divided cells in the retinal inner nuclear layer (INL) (FIG. 1). The results obtained by the calculation of the ratio of the divided cells are shown in FIG. 2. As is evident from the figure, the addition of Wnt3a caused a dramatic increase in the number of the divided cells in INL. As investigated by an immunological staining, most of these divided cells were identified as glutamine synthetase positive Muller cells (FIG. 3). As is evident from these results, the division of the Muller cells has been accelerated by Wnt3a.

Further, after 4 day culture with the addition of Wnt3a, the culture medium was changed, and the culture was then cultured for 1 week without Wnt3a. As a result, a part of the divided cells express Rhodopsin as a marker of the visual cells, which showed that the Muller cells differentiated into retinal nerve cells.

In this case, an effect of accelerating the differentiation to retinal cells was shown, but it was suggested that Wnt 3a also shows an effect of accelerating the differentiation of other neural cells. Wnt3a was expected to have a regeneration effect on a wide variety of neural cells.

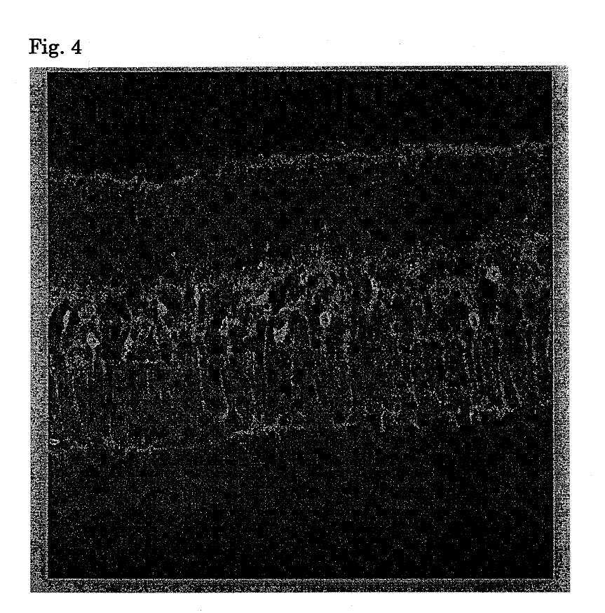

Further, an inhibitor of glycogen synthase kinase 3β(GSK-3β), AR-A014418(J, Biol. Chem., Vol, 278, No. 46, p45937-45945, 2003), was added to a culture medium for the retina organ culture and the effect thereof was examined. After incubating for 4 days with the addition of 5 μM AR-A014418 together with BrdU, as shown in FIG. 4, the number of BrdU-positive cells was increased. In contrast, the number of BrdU-positive cells in control without the addition of AR-AO14418 was small. From this fact, like the Wnt3a protein, the possibility that the GSK-3β inhibitor could be provided as a remedy for regeneration of nerve cells such as retinal cells was suggested.

### INDUSTRIAL APPLICABILITY

The remedy of the present invention can accelerate the regeneration of nerve cells and can be used to treat the disorders such as neurodegeneration disorder or neurologic dysfunction. In particular, the remedy of the present invention can be used to treat any disorder such as the retinitis pigmentosa, in which visual cells are irreversibly denatured, by regenerating the visual cells in the retina through activation of endogenous stem cells.

1. A remedy for nerve cell regeneration, comprising a Wnt3a protein or a Wnt3a signaling activator, wherein the nerve cell is a retinal cell.

2. (canceled)

3. The remedy according to claim 1, wherein the remedy is a therapeutic drug for retinitis pigmentosa, age-related macular degeneration, or cone dystrophy.

4. The remedy according to claim 1, wherein the Wnt3a protein comprises a sequence of amino acids 25 to 352 in SEQ ID No. 2 or a sequence of amino acids Nos. 25 to 352 in SEQ ID No. 2 with substitution, deletion, insertion, or addition of one or several amino acids and has-having an ability of regenerating nerve cells.

5. A method of regenerating a retinal cell, comprising administering a Wnt3a protein or a Wnt3a signaling activator to a nerve cell.

6. A method of regenerating a retinal cell, comprising administering a Wnt3a protein or a Wnt3a signaling activator to a retinal cell.

7. The remedy according to claim 3, wherein the Wnt3a protein comprises a sequence of amino acids 25 to 352 in SEQ ID No. 2 or a sequence of amino acids Nos. 25 to 352 in SEQ ID No. 2 with substitution, deletion, insertion, or addition of one or several amino acids and having an ability of regenerating nerve cells.


SEQUENCE LISTING (4 sequences)

SEQ ID NO: 1 - DNA, 1059 bp, Homo sapiens
  CDS (1)..(1056)
atggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtag

SEQ ID NO: 2 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 3 - DNA, 1059 bp, Mus musculus
  CDS (1)..(1056)
atggctcctctcggatacctcttagtgctctgcagcctgaagcaggctctgggcagctacccgatctggtggtccttggctgtgggaccccagtactcctctctgagcactcagcccattctctgtgccagcatcccaggcctggtaccgaagcagctgcgcttctgcaggaactacgtggagatcatgcccagcgtggctgagggtgtcaaagcgggcatccaggagtgccagcaccagttccgaggccggcgttggaactgcaccaccgtcagcaacagcctggccatctttggccctgttctggacaaagccacccgggagtcagcctttgtccatgccatcgcctccgctggagtagctttcgcagtgacacgctcctgtgcagagggatcagctgctatctgtgggtgcagcagccgcctccagggctccccaggcgagggctggaagtggggcggctgtagtgaggacattgaatttggaggaatggtctctcgggagtttgccgatgccagggagaaccggccggatgcccgctctgccatgaaccgtcacaacaatgaggctgggcgccaggccatcgccagtcacatgcacctcaagtgcaaatgccacgggctatctggcagctgtgaagtgaagacctgctggtggtcgcagccggacttccgcaccatcggggatttcctcaaggacaagtatgacagtgcctcggagatggtggtagagaaacaccgagagtctcgtggctgggtggagaccctgaggccacgttacacgtacttcaaggtgccgacagaacgcgacctggtctactacgaggcctcacccaacttctgcgaacctaaccccgaaaccggctccttcgggacgcgtgaccgcacctgcaatgtgagctcgcatggcatagatgggtgcgacctgttgtgctgcgggcgcgggcataacgcgcgcactgagcgacggagggagaaatgccactgtgttttccattggtgctgctacgtcagctgccaggagtgcacacgtgtctatgacgtgcacacctgcaagtag

SEQ ID NO: 4 - protein, 352 aa, Mus musculus
MAPLGYLLVLCSLKQALGSYPIWWSLAVGPQYSSLSTQPILCASIPGLVPKQLRFCRNYVEIMPSVAEGVKAGIQECQHQFRGRRWNCTTVSNSLAIFGPVLDKATRESAFVHAIASAGVAFAVTRSCAEGSAAICGCSSRLQGSPGEGWKWGGCSEDIEFGGMVSREFADARENRPDARSAMNRHNNEAGRQAIASHMHLKCKCHGLSGSCEVKTCWWSQPDFRTIGDFLKDKYDSASEMVVEKHRESRGWVETLRPRYTYFKVPTERDLVYYEASPNFCEPNPETGSFGTRDRTCNVSSHGIDGCDLLCCGRGHNARTERRREKCHCVFHWCCYVSCQECTRVYDVHTCK